(19) United States
(12) Patent Application Publication
(10) Pub. No.: US 2016/0184505 A1
(43) Pub. Date: Jun. 30, 2016

(54) PATIENT WARMING SYSTEM

(71) Applicant: Kenneth L. WILLEFORD, Little River, SC (US)

(72) Inventor: Kenneth L. WILLEFORD, Little River, SC (US)

(21) Appl. No.: 15/062,642

(22) Filed: Mar. 7, 2016

Related U.S. Application Data
(63) Continuation-in-part of application No. 11/657,171, filed on Jan. 24, 2007.
(60) Provisional application No. 60/762,240, filed on Jan. 26, 2006.

(51) Int. Cl. A61M 3/02; A61F 5/445; A61F 7/12

(52) U.S. Cl. CPC A61M 3/0258; A61F 7/12; A61F 5/445; A61F 2007/126; A61M 2210/1085

(57) ABSTRACT

A method of warming a patient during a surgical procedure may involve catheterizing the patient's bladder with a catheter that permits simultaneous flows into and out of the bladder. Fluid may be warmed and pumped into the bladder through the catheter, via a supply path, and simultaneously drained out of the bladder, via a return path. In steady state, there may exist a continuous air-fluid interface along one or more sections of a drain line of the return path having a negative slope with respect to the earth's gravitational field.

### CROSS REFERENCE TO RELATED APPLICATIONS

This application is a continuation-in-part of U.S. patent application Ser. No. 11/657,171, filed Jan. 24, 2007, which claims priority to U.S. Provisional Patent Application No. 60/762,240, filed Jan. 26, 2006, the contents of which are incorporated herein by reference.

### BACKGROUND

1. Field of the Invention

The present invention relates to the warming of a patient, e.g., for prevention of patient hypothermia during a surgical procedure.

2. Description of Related Art

Intraoperative hypothermia of surgical patients is a common problem with well documented adverse events. Unintentional intraoperative heat loss occurs as a result of low ambient temperatures in the operating room, open exposed wounds, administration of cool intravenous fluids, cool irrigating fluids, sterile preparation of the surgical site with fluids creating evaporative losses, and the effect of anesthetic agents that impair the body's ability to thermoregulate by the hypothalamus with concurrent vasodilation increasing heat loss and reduced metabolism with decreased heat production. (Patients have their skin exposed, washed with cold soap in a cold room and given medications that prevent the normal thermoregulatory mechanisms).

Physiologic effects of hypothermia include decreased oxygen tension in the blood, decreased metabolism, decreased drug biotransformation, impaired renal transport processes, altered membrane excitability, changes in cardiac rate and rhythm, central nervous system depression, hyperglycemia, and sympathetic nervous system stimulation.

Outcome studies have confirmed an increase in wound infection, myocardial infarction, need for post operative mechanical ventilation, probability for blood transfusion, and mortality in hypothermic versus normothermic patients.

Several methods are currently employed to prevent hypothermia in surgical patients. These include insulation of the patient, humidification of inspired respiratory gases, warming of intravenous fluids, and forced air warming blankets.

In the prior art of which I am aware, Lasheras et al., U.S. Pat. No. 6,648,906 disclosed an apparatus and a method for regulating patient temperature by irrigating the patient's bladder with a fluid. A catheter is inserted through the urethra and into the bladder, the catheter having at least two lumens; and a heated (or chilled) fluid is passed through a supply lumen of the catheter and into the bladder. The fluid is evacuated from the bladder through a return lumen of the catheter, and the quantity of urine flowing out of the bladder is monitored. The pressure of the fluid flowing into the supply lumen of the catheter, as well as to the return lumen, is also monitored.

As stated by Lasheras et al.:

Patients may require pre or post-operative cooling for a variety of reasons, including, for example, treatment of a malignant hypothermia crisis and induction of therapeutic hypothermia for neurosurgery.”
whereas my invention is intended to prevent hypothermia during a surgical procedure in the operating room (O.R.).

It is further noted that Lasheras et al. continually maintains a complete fluid column in the drain path, for the stated purpose, “because it has been noted that the pressure of the working fluid in the bladder must be maintained in order to maintain satisfactory heat transfer.” Lasheras et al. at col. 6, lines 30-33. However, this premise is incorrect, as will be discussed below, and unless monitored as in Lasheras et al., there may be risk of bladder rupture. Therefore, there may be advantages to a patient warming system that may avoid such risks, and which, at the same time, may be simpler than that of Lasheras et al.

### BRIEF SUMMARY OF ASPECTS OF THE DISCLOSURE

The present invention may provide a unique method of warming a patient to prevent hypothermia in the patient during a surgical procedure under operating room conditions without incurring the risk inherent in maintaining bladder pressurization. Such a method and apparatus may provide patient warming, via the bladder, while maintaining a continuous air-fluid interface along sections of the drain line from the bladder having a negative slope with respect to the gravitational field. This may result in distinct differences in pressure within the drain line, in comparison to Lasheras et al., and corresponding differences in pressure within the bladder, while maintaining adequate heat transfer rate.

My improved method includes the steps of: 1) catheterization of the patient's bladder to allow for simultaneous filling and draining of the bladder; 2) providing a pump and a fluid warmer; and 3) draining the warmed fluid and urine out of the bladder while maintaining a continuous air-fluid interface along sections of the drain line having a negative slope with respect to the gravitational field (of the earth), which may minimize potential complications of bladder rupture and/or thermal injury to the bladder.

Under such outflow conditions, the pressure of the fluid flowing through the return lumen ceases to be a potential problem, or at least may be greatly reduced.

These and other objects of the present invention will become apparent from a reading of the following specification taken in conjunction with the accompanying drawings.

### BRIEF DESCRIPTION OF THE DRAWINGS

FIG. 1 is a flow diagram of a method according to an aspect of the present invention.

FIGS. 2-5 are photographs of a simulation of a system showing a balloon to represent the patient's bladder with a catheter, a urinary drainage bag, a fluid management system tubing set and a blood/fluid warming system tubing set.

FIG. 6 is a conceptual block diagram of system components according to an aspect of this disclosure.

### DETAILED DESCRIPTION OF ASPECTS OF THE DISCLOSURE

The current invention utilizes continuous bladder irrigation with warm fluids to prevent hypothermia. This technique can affect patient temperature through two mechanisms. First, the bladder will become warm and dissipate this energy to adjacent tissues. More significantly, the arterial blood that supplies the bladder will leave through the veins at an elevated temperature and distribute this warmth systematically by the cardiovascular system. This mechanism can be significant as the bladder is quite a vascular organ with 6 supplying arteries and drained by a venous plexus. Tissue blood flow of the bladder has been measured as high as 74 milliliters per minute per one hundred grams of tissue.

Catheterization of the bladder is a routine and common procedure in surgical patients, particularly in longer operations where temperature regulation is a greater problem. The current prototype uses existing components and combines them in a unique manner to accomplish this task. It can be employed in any situation where the bladder is catheterized.

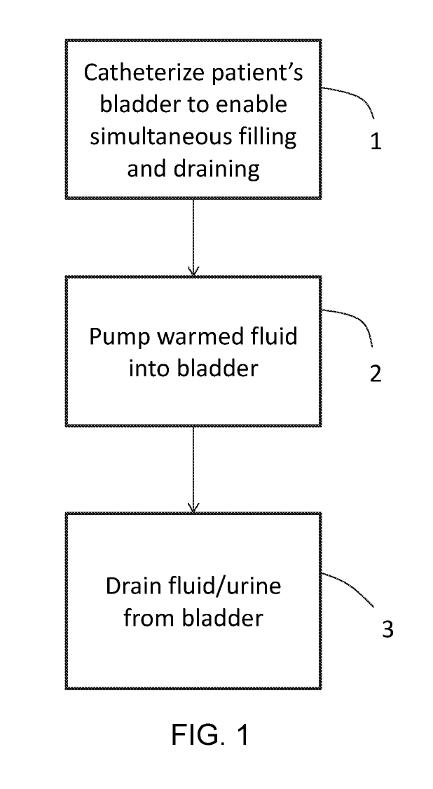

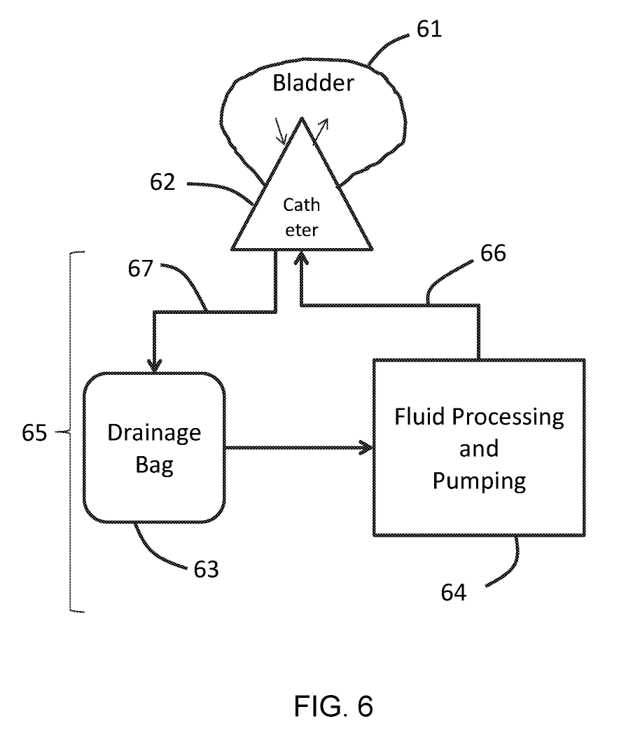

The method of the present invention is shown diagrammatically in FIG. 1. The patient's bladder may be catheterized 1 using a catheter that permits simultaneous filling and draining of the bladder. A warmed fluid, e.g., a saline solution, may be pumped into the bladder 2, and simultaneously, fluid and urine may drain 3 from the bladder. This will now be discussed in further detail in conjunction with FIG. 6.

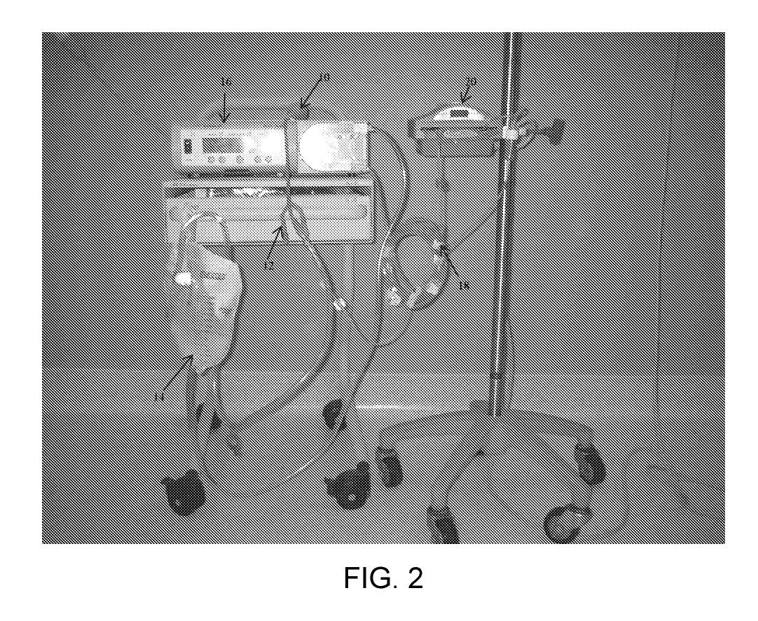

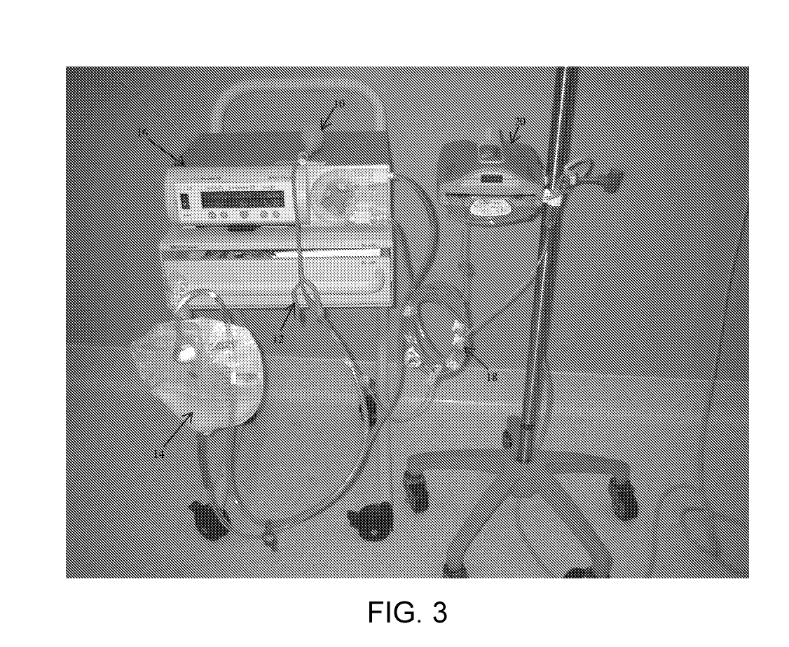

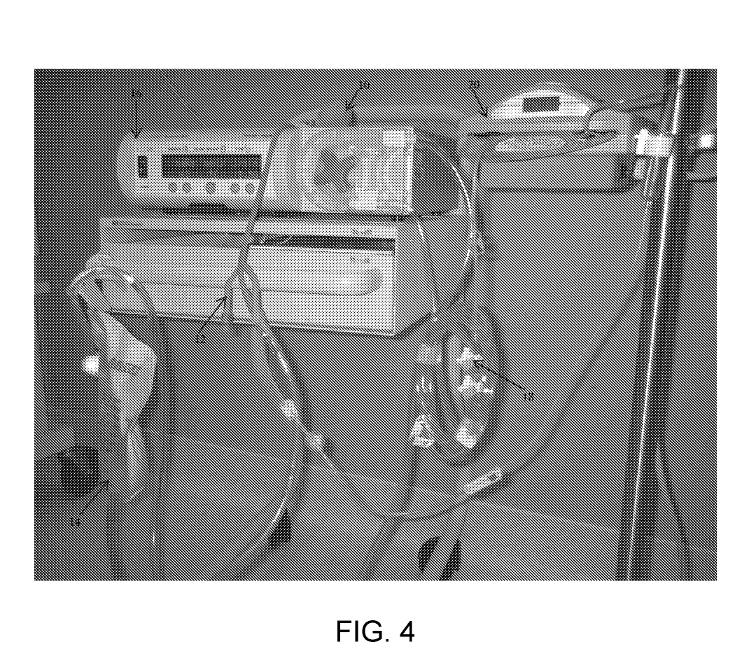

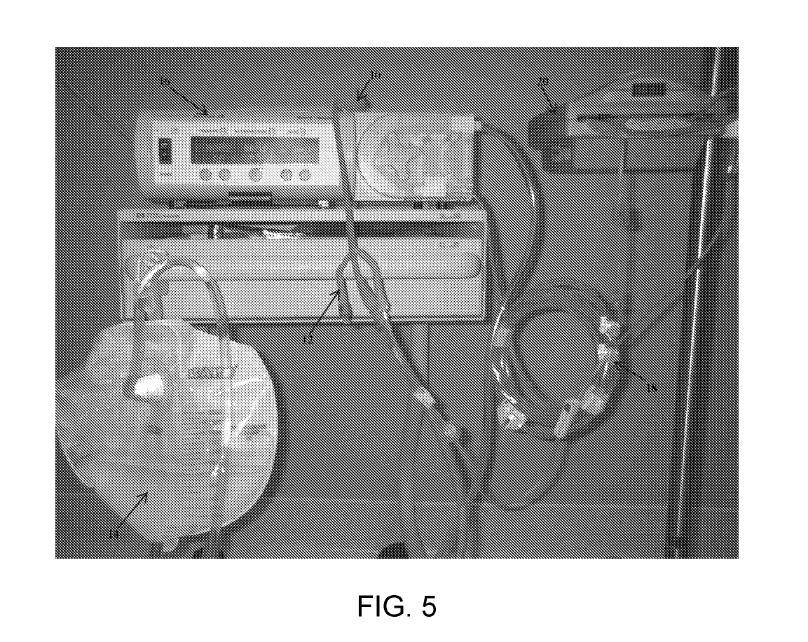

FIG. 6 shows a conceptual block diagram of apparatus that may be used to perform the operations of FIG. 1. The block diagram of FIG. 6 may include/represent the apparatus as shown in FIGS. 2-5, which will be discussed below. As shown in FIG. 6, a catheter 62 may be inserted into a patient's bladder 61. Catheter 62 may be configured to enable simultaneous flow of fluid into the bladder and out of the bladder, as indicated by the arrows leading into and out of catheter 62, within the bladder 61 (e.g., catheter 62 may be a three-way Foley catheter). Fluid draining out of the bladder through catheter 62 may pass through a drain line 67 of a return path 65 into a drainage bag (or other type of reservoir) 63, while fluid flowing into the bladder 61 may be pumped into the bladder 61, via catheter 62 and supply path 66, from a fluid processing and pumping component 64. The fluid processing and pumping component 64 may include, but is not limited to, sub-components for warming fluid, measuring urine output, filtering the fluid, pumping the fluid through supply path 66 and through catheter 62, etc. Such sub-components may be housed within a single housing/apparatus or may be two or more separate components that may be interconnected (e.g., as in FIGS. 2-5).

According to an aspect of this disclosure, the drainage bag/reservoir 63 may be physically located below the catheter 62, resulting in gravity being the dominant force by which urine drains from the bladder, via catheter 62 and drain line 67, into drainage bag/reservoir 63. As a result, bladder pressure is not necessary for drainage of the bladder 61.

According to an aspect of this disclosure, pressurization of the bladder is also not necessary to maintain a sufficient heat transfer rate. This is evident from the following example computations.

Heat capacity rate is a term used in thermodynamics to describe the quantity of heat a flowing fluid of a certain mass is able to absorb or release per unit temperature change per unit time. It is calculated as follows:

$$C = {c}_{p} \frac{d m}{d t}$$

where: C=heat capacity rate of the fluid of interest, cₚ=specific heat of the fluid of interest, and dm/dt=mass flow rate of the fluid of interest. The heat capacity rate calculation for the fluid delivered to the bladder may use, for example, a flow rate of 1.5 liters per minute, a mass density of 1.0 kilogram per liter of fluid, and a specific heat of 4.187 kilojoules per kilogram degree Celsius:

C=(4.139 kJ/kg ° C.)(1 kg/1 liter)(1.5 liters/minute)=6.21 kJ/minute ° C.

The potential increase in temperature for a 70 kg human, assuming, for example, a two-degree temperature difference between temperature of the fluid input to the bladder and fluid draining from the bladder, a two-hour period of use, and a specific heat of the human body of 3.5 kJ/kg ° C. may be calculated as:

[(6.21 kJ/minute ° C.)(2° C.)(120 minutes)]/[(3.5 kJ/kg ° C.)(70 kg)]=6.1° C.

This is calculated with an understanding of the context of a dynamic situation of simultaneous heat loss and heat delivery. These sample calculations verify the ability to warm a human through the use of bladder irrigation with warmed fluid under conditions of low flow (such as, but not limited to, an input flow rate of 1.5 liters per minute) such that there can exist a continuous air-fluid interface along sections of the drain line 65 having a negative slope with respect to the earth's gravitational field. That is, as distinguished from the prior art, e.g., Lasheras et al., the pressure of the working fluid in the bladder does not need to be maintained in order to maintain satisfactory heat transfer rate, as evidenced by these sample calculations, which allows for the improved design.

Turning back to FIG. 6, under such conditions, in a steady state (i.e., after initial starting and prior to shutdown), while the supply path 66 may contain a substantially continuous column/flow of fluid, the drain line 67 may, at the same time, contain a fluid flow that contains a continuous air-fluid interface along sections of the drain line 67 having a negative slope with respect to the earth's gravitational field. These conditions are shown in FIGS. 2-5. In general, the continuous air-fluid interface along sections of the drain line 67 having a negative slope with respect to the earth's gravitational field may generally be a continuous linear air-fluid interface (i.e., along sections of the drain line 67 having a negative slope with respect to the earth's gravitational field).

Because of the difference in the density of air vs. fluid, this may result in a distinction with respect to the pressure that is in the drain line compared to the situation with a complete fluid column in the drain path. This distinction in the pressure within the drain line then may result in a distinction with respect to the pressure in the bladder such that the pressure within the bladder of the instant application is distinctly reduced compared to the condition with a complete fluid column in the drain path, e.g., as in Lasheras et al. This result of the difference of the pressure in the drain line and pressure in the bladder is because of the difference in the density of air, which is 1.225 kg/m³, and the density of the fluid within the drain line, which is greater than the density of water (1000 kg/m³) because it is mixed with urine, which has a greater density due to dissolved solutes (which is measured as specific gravity). This distinction from the prior art may be accomplished with the physical design such that there is a continuous air-fluid interface along sections of the drain line with a negative slope with respect to the earth's gravitational field, as discussed above.

The factors/settings that may result in the above conditions may include, but are not limited to, pump flow rate (into supply path 66), catheter dimensions (e.g., the source and/or drain lumen dimensions), the dimension of the drain line 67, the slope of the drain line 67 between catheter 62 and drainage bag/reservoir 63, and the relative elevation of the drainage bag/reservoir 63 with respect to bladder 61 and/or catheter 62. These settings and factors may be determined empirically, and one of ordinary skill in the art would be capable of adjusting these settings and factors to result in the above conditions. Examples of settings, dimensions, and the like that may result in the above conditions are discussed below in connection with FIGS. 2-5.

FIGS. 2-5 show a simulated system according to aspects of this disclosure. In FIGS. 2-5, the balloon 10 represents the bladder. A Bard Urological Lubricath® 18 french foley catheter 12 with a 5 ml balloon (#0119L18) is inserted into the bladder. The bladder is drained by gravity into a Bard Urological® urinary drainage bag 14 (#154002). The drain tube of the urinary bag is connected to a Smith & Dyonics Access 15® (#7205699) fluid management system tubing set 16. The fluid and urine is then pumped through a Medex (#MX4312L) 3-way stopcock 18 and sequentially through a Ranger(® blood/fluid warming system tubing set 20 (#24200). The fluid continues through this system into the bladder via the 3-way foley.

The essential components include a 3-way foley to allow simultaneous filling and draining of the bladder 10, a pump, and a fluid warmer 20.

The Smith & Nephew Dyonics Access 15® (#7205699) fluid management system 16 is intended for use in orthopedic arthroscopy. It is a fluid pump where the flow rate and pressure can be adjusted. In this design, which maintains a continuous air-fluid interface along sections of the drain line having a negative slope with respect to the earth's gravitational field, there is distinctly reduced pressure in the bladder, which may otherwise predispose the patient to the complication of bladder rupture. The prototype has been successfully bench tested with a pressure of 60 millimeters of mercury and a flow rate of 1.5 liters per minute.

The Ranger® blood/fluid warming system 20 is used for warming intravenous fluids and blood transfusions. It is preset so the effluent temperature is 41° C. This is safe for intravenous fluids and would prevent complications of thermal injury. (The bladder can likely tolerate higher temperatures safely which would improve the effectiveness of this design for patient warming purposes; however the threshold temperature of the bladder lining to prevent injury has not been determined.)

The potential complications of bladder rupture and thermal injury have been addressed. There is an additional risk of infection, as with all procedures where the bladder is catheterized. All of the tubing components of this system are individually packaged in a sterile fashion and will need to be assembled with attention to sterile technique.

An additional consideration is the measurement of urine output. This is typically measured as the total fluid collected in the bag. This patient warming system will need to be primed with sterile fluid (saline), and the urine output measured as the increase in total fluid within the system.

Obviously, many modifications may be made without departing from the basic spirit of the present invention. Accordingly, it will be appreciated by those skilled in the art that within the scope of the appended claims, the invention may be practiced other than has been specifically described herein.

I claim:

1. A method of warming a patient, the method comprising:
catheterizing the patient's bladder using a catheter configured to enable simultaneous filling and draining of the bladder;
warming a fluid to be pumped into the bladder to provide warmed fluid at a temperature sufficient to warm the patient without causing thermal injury to the bladder; and
continuously pumping the warmed fluid into the bladder through the catheter, via a supply path, and simultaneously draining fluid and urine from the bladder through the catheter, via a drain line, into a receptacle located at a height physically below the bladder and the catheter;
wherein, in steady state, there exists a continuous air-fluid interface along one or more sections of the drain line having a negative slope with respect to the earth's gravitational field.

2. The method of claim 1, further comprising setting a flow rate of the continuously pumping to enable the continuous air-fluid interface along one or more sections of the drain line having a negative slope with respect to the earth's gravitational field to be maintained in the drain line.

3. The method of claim 1, further comprising dimensioning input and output lumens of the catheter and the drain line to facilitate the existence of the continuous air-fluid interface along one or more sections of the drain line having a negative slope with respect to the earth's gravitational field.

4. The method of claim 1, further comprising measuring fluid and urine drained from the bladder to monitor urine output of the patient.

5. The method of claim 4, further comprising priming the patient warming system with sterile fluid.

6. A patient warming system comprising:
a fluid warming and pumping device configured to provide warmed fluid at a temperature sufficient to warm a patient through the patient's bladder without causing thermal injury to the patient's bladder and configured to continuously pump the warmed fluid into the patient's bladder through a urinary catheter, via a supply path, at a flow rate;
a fluid receptacle coupled to an input of the fluid warming device and to an output from the urinary catheter to enable simultaneous draining of fluid and urine from the patient's bladder, via a return path, while the fluid warming and pumping device is continuously pumping the warmed fluid into the patient's bladder, wherein the receptacle is located at a height physically below the urinary catheter and the patient's bladder; and
a drain line of the return path configured to couple the output of the urinary catheter to the fluid receptacle, wherein one or more sections of the drain line have a negative slope with respect to the earth's gravitational field,
wherein, in steady state, there exists a continuous air-fluid interface along at least one of the one or more sections of the drain line having a negative slope with respect to the earth's gravitational field.

7. The patient warming system of claim 6, wherein the flow rate is set such that there exists the continuous air-fluid interface along the at least one of the one or more sections of the return path having a negative slope with respect to the earth's gravitational field.

8. The patient warming system of claim 6, wherein dimensions of an input lumen and an output lumen of the urinary catheter and of the drain line are chosen to enable the existence of the continuous air-fluid interface along the at least one of the one or more sections of the drain line having a negative slope with respect to the earth's gravitational field.

9. The patient warming system of claim 6, wherein the fluid warming and pumping device includes:
a fluid warming device coupled to an output of the fluid receptacle; and
a fluid pumping device coupled to an output of the fluid warming device.

10. The patient warming system of claim 6, wherein the fluid receptacle is a drainage bag.
